(19) United States
(12) Patent Application Publication
(10) Pub. No.: US 2023/0364012 A1
(43) Pub. Date: Nov. 16, 2023

(54) STABLE OPHTHALMIC COMPOSITION OF LOTEPREDNOL

(71) Applicant: MANKIND PHARMA LTD., New Delhi (IN)

(72) Inventors: Sandeep SINGH, Manesar (IN); Ankush GUPTA, Manesar (IN); Hanumant GAMBHIRE, Manesar (IN); P.V.S NARASIMHAM, Manesar (IN); Anil KUMAR, Manesar (IN)

(21) Appl. No.: 18/316,644

(22) Filed: May 12, 2023

(30) Foreign Application Priority Data
May 13, 2022 (IN) 202211027609

(51) Int. Cl. A61K 9/00; A61K 31/567; A61K 47/32; A61K 47/18; A61K 47/10; A61K 47/36; A61K 9/10

(52) U.S. Cl. CPC A61K 9/0048; A61K 31/567; A61K 47/32; A61K 47/186; A61K 47/183; A61K 47/10; A61K 47/36; A61K 9/10

(57) ABSTRACT

The present invention relates to a stable ophthalmic composition comprising loteprednol or its pharmaceutically acceptable salt thereof, one or more suspending agents, and optionally one or more excipients.

### FIELD OF THE INVENTION

The present invention relates to stable ophthalmic compositions comprising Loteprednol and a method of preparation thereof.

### BACKGROUND OF THE INVENTION

Loteprednol etabonate is a new generation glucocorticoid drug, used for the treatment of local and systemic inflammatory reactions after cataract resection and artificial lens implantation, and is the first drug largely suitable for the postoperative ocular inflammation. Loteprednol etabonate is also suitable for the treatment of corticosteroid-sensitive inflammation such as inflammation of the eyelids and bulbar conjunctivitis, uveitis, cornea and anterior segment of the eye. The FDA approved dosage forms of loteprednol etabonate are mainly suspension eye drops, ointments and ophthalmic gels.

Lotemax® (loteprednol etabonate (LE) ophthalmic gel, 0.5% LE) (Bausch & Lomb Incorporated) contains 5 mg/g of loteprednol etabonate, as a sterile preserved ophthalmic gel suspension, and has proven effective for the treatment of post-operative inflammation and pain following ocular surgery. Lotemax® ophthalmic gel, 0.5% LE, contains boric acid, edetate disodium dihydrate, glycerin, polycarbophil, propylene glycol, sodium chloride, tyloxapol, water, and sodium hydroxide to adjust pH between 6 and 7, and is preserved with benzalkonium chloride (BAK) 0.003%.

Lotemax® ophthalmic gel, 0.38% LE ophthalmic gel, 0.5% LE) (Bausch & Lomb Incorporated) contains 0.38 mg/g of sub-micronized loteprednol etabonate, boric acid, edetate disodium dihydrate, glycerin, hypromellose, poloxamer, polycarbophil, propylene glycol, sodium chloride, water for injection, and sodium hydroxide to adjust pH of between 6 and 7, and is preserved with benzalkonium chloride (BAK) 0.003%.

U.S. Pat. No. 10,596,107 B2 discloses an ophthalmic suspension which includes an active ingredient suspended in a formulation vehicle, a suspending agent and a non-ionic cellulose derivative. The suspension may be administered to a patient for treating an ophthalmic inflammatory condition.

U.S. Pat. No. 8,999,938 B2 discloses an ophthalmic drug delivery composition comprising: an ophthalmic drug; one or more nonionic surfactants preferably Poloxamer 407, one or more non-Newtonian high blend viscosity enhancing, non-gelling agents selected from carboxymethyl cellulose, hydroxypropyl methyl cellulose, hydroxypropyl cellulose or a combination thereof.

U.S. Pat. No. 9,421,265 B2 claims a multi-dose ophthalmic composition, comprising polyols, effective amount of borate, a therapeutic agent, carboxyvinyl polymer as a suspending agent.

Chinese Patent Application No. 103565741 A discloses a glucocorticoid ophthalmic water suspension, suspending agent is selected from hyaluronate sodium, one or more in sodium carboxymethyl cellulose, hydroxypropyl methylcellulose, hydroxypropyl cellulose, polyvinyl alcohol, carboxy vinyl polymer, polyvinyl pyrrolidone, carbomer.

US Patent Application No. 20210290527 A1 discloses an ocular composition, comprising: a shear-thinning hydrogel comprising hyaluronic acid, wherein the hyaluronic acid is at a concentration of about 3 to about 10 mg/ml and is covalently cross-linked; and a therapeutic agent, wherein the therapeutic agent has a solubility in water of less than about 1.5 mg/ml.

Ophthalmic drug efficacy is severely limited by non-compliance. Compliance is adversely affected by the reduced comfort, irritation, and transient quality of vision loss, which lasts minutes to tens of minutes, that is common to many drugs. In particular, these adverse effects are caused by suspensions commonly used for highly lipophilic drugs or the requirement of very high topical concentrations for highly hydrophilic drugs.

The fundamental challenges of ophthalmic delivery vehicles are to improve comfort; minimize visual blur on instillation; increase drug solubility; increase drug residence time and permeation through the cornea to achieve greater intraocular delivery; reduce systemic drug absorption; and cause minimal local adverse effect. Unfortunately, these objectives are not met by current ophthalmic formulations.

Thus, there still exists a need in art for stable ophthalmic compositions of the loteprednol that are stable, have required penetration, and retention/residence time in an ocular tissues.

### SUMMARY OF THE INVENTION

The present invention relates to a stable ophthalmic composition comprising loteprednol, one or more suspending agents, and optionally one or more excipients.

Another aspect of the present invention provides a stable ophthalmic composition comprising loteprednol, one or more suspending agent, one or more viscosity modifiers, and optionally one or more excipients.

Another aspect of the present invention provides a stable ophthalmic composition comprising 0.05%-0.5% by weight of loteprednol having particle size D₅₀<1 μm, one or more suspending agent, and optionally one or more excipients

Another aspect of the present invention provides a stable ophthalmic composition comprising 0.38% by weight of loteprednol having particle size D₅₀<1 μm and D₉₀<3 μm, one or more suspending agents, one or more viscosity modifiers, and one or more buffering agent, one or more surfactant, and optionally a preservative, wherein the pH of the composition is in the range of 4-9.

The present invention further relates to a process for preparation of a stable ophthalmic composition comprising 0.05%-0.5% by weight loteprednol.

### DETAILED DESCRIPTION OF THE INVENTION

The present invention relates to a stable ophthalmic composition comprising loteprednol or its pharmaceutically acceptable salt thereof, one or more suspending agents, and optionally one or more excipients.

As used in this specification, the singular forms “a”, “an”, and “the” include plural references unless the context clearly dictates otherwise. Thus for example, a reference to “a method” or “a process” includes one or more methods, one or more processes and/or steps of the type described herein and/or which will become apparent to those persons skilled in the art upon reading this disclosure and so forth.

The term “pharmaceutical composition” or “composition” or “ophthalmic composition” or “dosage form” or as used herein synonymously include solution, suspension, gel, gel forming suspension (in-situ gels), ointment, lotion or any other suitable dosage form meant for administration to eye. Preferably, composition is a suspension, a gel, or a gel forming suspension.

The term “loteprednol” as used in the context of the present invention refers to loteprenol or loteprednol etabonate. Loteprednol etabonate is a known compound and can be synthesized by methods disclosed in U.S. Pat. No. 4,996,335, the entire contents of which are hereby incorporated by reference in the present specification.

According to various aspects, the concentration of Loteprednol etabonate in the formulation vehicle is in the range from 0.1 wt. % to 2 wt. %, or from 0.14 wt. % to 1.5 wt. %, or from 0.2 wt. % to 1 wt. %, or from 0.2 wt. % to 0.5 wt. %. A specific concentration of Loteprednol etabonate is 0.38 wt %.

The present invention provides a stable ophthalmic composition comprising loteprednol, wherein the loteprednol or its pharmaceutically acceptable salt thereof has particle size less D₅₀<5 μm, preferably D₅₀<3 μm, more preferably D₅₀<1 μm. D₅₀ is the particle diameter below which particles having 50% of the cumulative volume of all the particles are present. D₉₀<10 μm, preferably D₉₀<5 μm, more preferably D₉₀<3 μm. D₉₀ is the particle diameter below which particles having 90% of the cumulative volume of all the particles are present.

The term “stable” according to present invention refers to a storage stable composition with no signs of particle agglomeration, or flocculation for at least six months, for at least one year, or for at least two years. The term “stable” also refers to a composition in which is stable on exposure to 40° C.±5° C./75%±5% RH for a period of six months or 25° C.±5° C./60%±5% RH for a period of at least six months or at least 12 months.

A suitable suspending agent may comprise synthetic, semisynthetic, natural suspending agents, or mixtures thereof. Examples include but not limited to gellan gum, xanthan gum, guar gum, chitosan, alginic acid and its salts, xyloglucan, pectin, hyaluronic acid-agar, carrageenan, shellac, and hyaluronic acid derivatives, cellulose derivatives, such as methyl cellulose, ethyl cellulose, hydroxyl ethyl cellulose, polyethylene glycols (such as polyethylene glycol 300, polyethylene glycol 400, polyethylene glycol 6000), carboxymethyl cellulose, carboxymethylcellulose, hydroxypropylmethyl cellulose, a carboxyvinyl polymer such as polycarbophil, cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), poly(N-isopropylacrilamide), poly(N-isopropylacrilamide) monomer crosslinked with N,N-methylenebisacrylamide, polyethylene glycol (PEG)-conjugated-poly(N-isopropylacrilamide), poly(hydroxyethylmethacrylate)-conjugated-poly(N-isopropylacrilamide), poly(N-isopropylacrilamide)-block-poly(methyl methacrylate)-(poly(acrylic acid), polyvinylacetal diethylaminoacetate, poly(2-hydroxyethylmethacrylate-co-2-(diisopropylamino)ethyl methacrylate), Poly(2-hydroxyethyl methacrylate), gelatine methacrylate (GelMA), PLGA-PEG-PLGA triblock copolymer hydrogels, poly(ethylene glycol) acrylate, poly(ethylene glycol) diacrylate, poly(ethylene glycol) methacrylate, polycaprolactone-polylactic acid, hydroxyethyl methacrylate-methacrylic acid, Polyvinyl alcohol (PVA) and Polyvinylpyrrolidone (PVP), poly(amino acids), methyl methacrylate cross-linked poly(vinyl alcohol), Polyglycolic acid-polycaprolactone-polyethylene glycol-polycaprolactone-polyglycolic acid (PGA-PCL-PEG-PCL-PGA), polylactic acid-polycaprolactone-polyethyleneglycol-polycaprolactone-polylactic acid (PLA-PCL-PEG-PCL-PLA), poly(N-isopropylacrylamide)-poly(ethylene glycol)-poly(ε-caprolactone), poly (N,N-diethylacrylamide) and poly(N-isopropylacrylamide), poly(N-n-propylacrylamide), ethylene vinyl acetate polymer, polyalkyl cyanoacrylate, hydroxypropyl methacrylate mixture of tocopheryl acetate:medium-chain triglycerides, polyoxyethylene hydrogenated castor oil, or combinations thereof. The suspending agent(s) may be present in an amount of about 0.05 to about 10 wt %.

The stable ophthalmic composition of the present invention may further comprise viscosity modifiers.

The viscosity modifier includes but is not limited to glycerol, propylene glycol, polymeric polyols, such as, polyethylene glycol (such as polyethylene glycol 300, polyethylene glycol 400, polyethylene glycol 6000), dextrans such as dextran 70, water soluble proteins such as gelatin, polyvinyl alcohols, polyvinylpyrrolidones, cellulose derivatives, carbomers, gums such as gellan gum, xanthan gum, guar gum, chitosan, alginic acid and its salts, xyloglucan, pectin, hyaluronic acid-agar, carrageenan, shellac, and hyaluronic acid derivatives, dextrans, polyvinyl alcohol, polyacrylic acids, povidone such povidone K90, and polysaccharides such as hyaluronic acid and its salts and chondroitin sulfate and its salts, or combinations thereof. The viscosity modifiers may be present in an amount of about 0.05 to about 10 wt %.

In one of the embodiments, the ophthalmic composition of the present invention does not comprise non-ionic cellulosic derivatives such as hydroxypropylmethyl cellulose (“HPMC”), hydroxypropylcellulose (“HPC”), methyl cellulose (MC), hydroxyethyl cellulose (HEC), ethyl cellulose (EC), and microcrystaliine cellulose (MCC).

According to another embodiment of the present invention, the ophthalmic composition does not comprise carboxyvinyl polymer.

According to another embodiment of the present invention, the ophthalmic composition does not comprise polycarbophil.

According to one more embodiment of the present invention the ophthalmic composition does not comprise non-ionic cellulosic derivatives and polycarbophil.

The term “excipient(s)” or “pharmaceutically acceptable excipient(s)” means a component of a composition that is not an active ingredient, and includes but not limited to surfactants, tonicity agents, buffers, preservatives, chelating agents, pH modifying/adjusting agents.

A suitable buffering system may be included in the formulation to prevent pH change/shift under storage conditions. Buffers include but is not limited to phosphate buffer, acetate buffer, citrate buffer, succinate buffer, borate buffers, tris HCl and amino acids such as glycine, aspartate, histidine, cysteine, tyrosine, phenylalanine, proline, arginine, threonine, serine, valine, isoleucine, lycine, and glutamine. The particular concentration of the buffer will differ, depending on the specific agent employed.

The compositions will typically have a pH in the range of 4 to 9, preferably 5.5 to 8.5, and most preferably 6 to 7. In more specific embodiment, the initial pH of the composition is adjusted to 6.5±0.2.

The tonicity agent includes but is not limited to are sodium chloride, potassium chloride, magnesium chloride, calcium chloride, non-ionic diols such as glycerol and propylene glycol, dextrose and/or mannitol, sorbitol. Amount of tonicity agent will vary, depending on the certain agent to be added. The tonicity agent(s) is preferably used in an amount of about 0.05% to about 10 wt %.

Surfactant includes but is not limited to poloxamers, polysorbates, cyclodextrins, alkylaryl polyethers, polyoxyethyleneglycol alkyl ethers, tyloxapol, and polyoxyls. Poloxamers are nonionic triblock copolymers composed of a central hydrophobic chain of polyoxypropylene (poly(propylene oxide)) flanked by two hydrophilic chains of polyoxyethylene (poly(ethylene oxide)). Polysorbates are oily liquids derived from ethoxylated sorbitan esterified with fatty acids. Cyclodextrins are composed of 5 or more α-D-glucopyranoside units linked together at position 1 and 4. Polyoxyls are a mixture of mono- and diesters of stearate and polyoxyethylene diols. Preferred embodiments include but are not limited to poloxamer 188 (such as Pluronic® F-68) and poloxamer 407 (such as Pluronic® F127); polysorbate 20, polysorbate 60, polysorbate 80, tyloxapol, Brij® 35, Brij® 78, Brij® 98 and Brij® 700, Span® 20, Span® 40, Span® 60, Span® 80; cyclodextrins-2-HP25 cyclodextrin, ionically charged (e.g. anionic) beta-cyclodextrins with or without a butyrated salt (Captisol®; (sulfobutylether β-cyclodextrin, Captisol is a registered trademark of Cydex Pharmaceuticals), hydroxypropyl-gamma-cyclodextrin, gamma cyclodextrin; and polyoxylspolyoxyl 40 stearate, polyoxyl 30 castor oil, polyoxyl 35 castor oil, and polyoxyl 40 hydrogenated castor oil; Hydrogenated Castor oil (or PEG (40 Hydrogenated castor oil) (HCO-40) or combinations thereof. The surfactant(s) may be used in an amount of about 0.005 to about 5.0% wt.

For suspension, gels, or gel forming suspensions, it is desirable to have the viscosity to be sufficient to keep a therapeutic agent suspended for a substantial period of time. The viscosity of the suspension gel is typically less than 1000 cps, more typically less than 500 cps.

The compositions of the present invention may include a preservative. Suitable preservative includes but is not limited to benzalkonium chloride (BAC), chlorhexidine gluconate, benzethonium chloride, cetyl pyridinium chloride, benzyl bromide, phenylmercury nitrate, phenylmercury acetate, thiomerosal, merthiolate, phenylmercuryborate, methylparaben, propylparaben, sorbic acid, potassium sorbate, sodium benzoate, sodium propionate, ethyl p-hydroxybenzoate, butyl-p-hydroxybenzoate, chlorobutanol, sorbic acid, poly quaternary ammonium compounds, or mixtures thereof. The preservative(s) may be used in an amount of about 0.005 to about 2.0% wt.

According to one embodiment of the present invention the ophthalmic composition is free of preservative.

As to chelating agents, any suitable pharmaceutically acceptable chelating agent can be used. Examples include but not limited ethylenediaminetetraacetic acid and metal salts thereof, such as disodium edetate, trisodium edetate, tetrasodium edetate or mixtures thereof. The chelating agent(s) may be added in an amount of about 0.005 to about 5.0 wt %.

The pH modifying agent is typically a mineral acid or metal hydroxide base preferably selected from the group of potassium hydroxide, sodium hydroxide, hydrochloric acid, or mixtures thereof, and preferably sodium hydroxide and/or hydrochloric acid. These acidic and/or basic pH modifying agents are added to adjust the formulation to the target pharmaceutically acceptable pH range. Hence it may not be necessary to use both acid and base—depending on the formulation, the addition of one of the acid or base may be sufficient to bring the mixture to the desired pH range.

According to one embodiment, the stable ophthalmic composition of the present invention comprises:
Loteprednol in amount of about 0.05% w/w to about 0.5% w/w of the pharmaceutical composition;
suspending agent(s) in amount of about 0.1% w/w to about 5.0% w/w of the pharmaceutical composition;
viscosity modifier(s) in amount of about 0.1% w/w to about 5% w/w of the pharmaceutical composition;
preservative(s) in an amount of about 0.001% w/w to 0.05% w/w of the pharmaceutical composition;
surfactant(s) in an amount of about 0.05% w/w to 1.0% w/w of the pharmaceutical composition;
chelating agent(s) in amount of about 0.01% w/w to about 0.1% w/w of the pharmaceutical composition;
tonicity adjusting agent(s) in an amount of about 0.01% w/w to about 0.5% w/w of the pharmaceutical composition, wherein the composition is having a pH in the range of 6.0-7.0.

According to one embodiment, the stable ophthalmic composition of the present invention comprises:
Loteprednol etabonate in amount of about 0.38% w/w of the pharmaceutical composition;
carboxyvinyl polymer in amount of about 0.375% w/w of the pharmaceutical composition;
viscosity modifiers selected from glycerol, propylene glycol, polyethylene glycol, polyvinyl alcohol, polyvinylpyrrolidone or combinations thereof, in amount of about 0.5% % w/w to about 3% w/w of the pharmaceutical composition;
benzalkonium chloride in an amount of about 0.003% w/w of the pharmaceutical composition;
poloxamer 407 in an amount of about 0.2% w/w of the pharmaceutical composition;
edetate disodium in amount of about 0.055% w/w of the pharmaceutical composition;
sodium chloride in an amount of about 0.05% w/w of the pharmaceutical composition, wherein the composition is having an initial pH of 6.5±0.2.

The present invention also provides a process for the preparation of a stable ophthalmic composition of the present invention, the process comprises:
preparing solution phase comprising one or pharmaceutically acceptable excipients;
preparing dispersion phase comprising loteprednol, gelling agent, and optionally surfactant(s);
mixing both phases to obtain a suspension.

Loteprednol with particle size used in the present invention may be obtained by methods generally known in the art. For example, an aqueous slurry, containing the active and the formulation vehicle, is subjected to fluid micronization or bead milling, for a suitable time to obtain the desired particle size. Representative techniques for particle size reduction are fluid micronization and bead milling. In alternate embodiment, high pressure homogenizer and high shear homogenizer are used for particle size reduction of Loteprednol.

In one embodiment, the dispersion phase is prepared by milling or homogenising loteprednol together with gel phase comprising suspending agent(s) and preferably also comprising surfactant(s).

In one embodiment, the dispersion phase is prepared by milling or homogenising loteprednol together with surfactant phase consisting essentially of surfactant(s).

Another embodiment of the present invention provides a method of treatment of local and systemic inflammatory reactions after cataract resection, artificial lens implantation and postoperative ocular inflammation.

The present invention is further illustrated by the following examples which are provided merely to be exemplary of the invention and don't limit the scope of the invention. Certain modifications and equivalents will be apparent to those skilled in the art and are intended to be included within the scope of the present invention.

### EXAMPLES

A representative gel, suspension, or gel forming suspension of this invention comprises or consists essentially of, or consists of the following composition:

| Ingredient | % by weight of total composition |
|---|---|
| API micronized | 0.05-0.5% |
| Suspending Agent | 0.01-10% |
| Viscosity modifier | 0-10% |
| Preservative | 0-5% |
| Chelating Agent | 0-5% |
| Tonicity Agent | 0-10% |
| Surfactant | 0-5% |
| Buffer | 0.001-10% |
| Water as Diluent | q.s. to 100% |
| pH Adjuster | q.s. to pH of 6-8 |

### Example 1

According to various aspects, gel, suspension, or gel forming suspension comprises or consists essentially of, or consists of the following composition:

| Ingredient | mg/ml |
|---|---|
| Loteprednol etabonate | 3.8 |
| D₅₀ < 1 μm and D₉₀ < 3 μm | |
| Polycarbophil | 3.75 |
| Polyvinyl pyrrolidinone (PVP) | 2.50 |
| Benzalkonium chloride | 0.03 |
| Edetate Disodium Dihydrate | 0.55 |
| Sodium Chloride | 0.50. |
| Tyloxapol | 2.00 |
| Propylene glycol | 4.40 |
| Boric acid | 5.00 |
| Water as Diluent | q.s. to 1 ml |
| pH Adjuster | q.s. to pH of 6-7 |

Process:
1. Dispensed all ingredients.
2. Prepared and milled the slurry comprising Loteprednol etabonate, polycarbophil, and Tyloxapol in water to achieve a dispersion phase with desired particle size.
3. Added Benzalkonium chloride, Edetate Disodium Dihydrate, Sodium Chloride, and Boric acid to water to obtain a solution phase.
4. Mixed dispersion phase of step 2 and solution phase of step 3 under continuous stirring, followed by addition of propylene and PVP to adjust the viscosity, as required, to obtain a suspension.
5. Adjusted the pH of the suspension.

### Example 2

According to various aspects, gel, suspension, or gel forming suspension comprises or consists essentially of, or consists of the following composition:

| Ingredient | mg/ml |
|---|---|
| Loteprednol etabonate | 3.8 |
| D₅₀ < 1 μm and D₉₀ < 3 μm | |
| Carbomer 947 | 3.00 |
| Carboxymethyl cellulose | 3.00 |
| Benzalkonium chloride | 0.03 |
| Edetate Disodium Dihydrate | 0.55 |
| Sodium Chloride | 0.50. |
| Poloxamer 188 | 2.00 |
| Mannitol | 4.40 |
| Boric acid | 5.00 |
| Water as Diluent | q.s. to 1 ml |
| pH Adjuster | q.s. to pH of 6-7 |

Process:
1. Dispensed all ingredients.
2. Prepared and milled the slurry comprising Loteprednol etabonate, Carbomer 947 and Poloxamer 188 in water to achieve a dispersion phase with desired particle size.
3. Added Benzalkonium chloride, Edetate Disodium Dihydrate, mannitol, Sodium Chloride, and Boric acid to water to obtain a solution phase.
4. Mixed dispersion phase of step 2 and solution phase of step 3 under continuous stirring, followed by addition of carboxymethyl cellulose to adjust the viscosity, as required, to obtain a suspension.
5. Adjusted the pH of the suspension.

### Example 3

According to various aspects, gel, suspension, or gel forming suspension comprises or consists essentially of, or consists of the following composition:

| Ingredient | mg/ml |
|---|---|
| Loteprednol etabonate | 3.8 |
| D₅₀ < 1 μm and D₉₀ < 3 μm | |
| Hydroxypropyl methyl cellulose | 3.00 |
| Xanthan Gum | 3.00 |
| Benzalkonium chloride | 0.03 |
| Edetate Disodium Dihydrate | 0.45 |
| Sodium Chloride | 0.75. |
| Poloxamer 407 | 1.80 |
| Glycerin | 3.50 |
| Propylene glycol | 1.90 |
| Boric acid | 4.50 |
| Water as Diluent | q.s. to 1 ml |
| pH Adjuster | q.s. to pH of 6.5 |

Process:
1. Dispensed all ingredients.
2. Prepared and milled the slurry comprising Loteprednol etabonate and poloxamer 407 in water to achieve a dispersion phase with desired particle size.
3. Added Benzalkonium chloride, Edetate Disodium Dihydrate, mannitol, Sodium Chloride, and Boric acid to water to obtain a solution phase.
4. Added xanthan gum, hydroxypropyl methyl cellulose, glycerine, and propylene glycol to obtain a gel phase.
5. Mixed the dispersion phase of step 2, solution phase of step 3, and gel phase of step 4 under continuous stirring, to obtain a suspension.
6. Adjusted the pH of the suspension.

### Example 4

According to various aspects, gel, suspension, or gel forming suspension comprises or consists essentially of, or consists of the following composition:

| Ingredient | mg/ml |
|---|---|
| Loteprednol etabonate | 3.8 |
| D₅₀ < 1 μm and D₉₀ < 3 μm | |
| Gellan gum | 3.50 |
| Carbomer 947 | 2.75 |
| Benzalkonium chloride | 0.05 |
| Edetate Disodium Dihydrate | 0.45 |
| Sodium Chloride | 0.50. |
| Polysorbate 80 | 2.00 |
| Sorbitol | 4.40 |
| Boric acid | 5.00 |
| Water as Diluent | q.s. to 1 ml |
| pH Adjuster | q.s. to pH of 6.5 |

Process:
1. Dispensed all ingredients.
6. Prepared and milled the slurry comprising Loteprednol etabonate, Carbomer 947, gellan gum, and polysorbate 80 in water to achieve a dispersion phase with desired particle size.
2. Added Benzalkonium chloride, Edetate Disodium Dihydrate, sorbitol, Sodium Chloride, and Boric acid to water to obtain a solution phase.
3. Mixed the dispersion phase of step 2 and solution phase of step 3 under continuous stirring, to obtain a suspension.
4. Adjusted the pH of the suspension.

### Example 5-7

According to various aspects, gel, suspension, or gel forming suspension comprises or consists essentially of, or consists of the following composition:

| | Example 5 | | Example 6 | | Example 7 | |
|---|---|---|---|---|---|---|
| Ingredients | mg/g | % w/w | mg/g | % w/w | mg/g | % w/w |
| Loteprednol | 3.80 | 0.380 | 3.80 | 0.380 | 3.80 | 0.380 |
| Etabonate | | | | | | |
| Benzalkonium | 0.030 | 0.003 | 0.030 | 0.003 | 0.030 | 0.003 |
| Chloride | | | | | | |
| Povidone | 6.0 | 0.60 | — | — | — | — |
| Polyvinyl Alcohol | — | — | 10.0 | 1.00 | | |
| Polyethylene | — | — | — | — | 8.0 | 0.80 |
| Glycol 400 | | | | | | |
| Poloxamer (407) | 2.00 | 0.200 | 2.00 | 0.200 | 2.00 | 0.200 |
| Edetate disodium | 0.550 | 0.055 | 0.550 | 0.055 | 0.550 | 0.055 |
| (Dihydrate) | | | | | | |
| Glycerin (100%) | 8.800 | 0.880 | 8.800 | 0.880 | 8.800 | 0.880 |
| Propylene Glycol | 4.400 | 0.440 | 4.400 | 0.440 | 4.400 | 0.440 |
| Boric Acid | 5.00 | 0.500 | 5.00 | 0.500 | 5.00 | 0.500 |
| Polycarbophil | 3.75 | 0.375 | 3.75 | 0.375 | 3.75 | 0.375 |
| Sodium Chloride | 0.50 | 0.050 | 0.50 | 0.050 | 0.50 | 0.050 |
| Sodium Hydroxide | q.s. to | q.s. to | q.s. to | q.s. to | q.s. to | q.s. to |
| | adjust | adjust | adjust | adjust | adjust | adjust |
| | pH | pH | pH | pH | pH | pH |
| Water for Injection | q.s. | q.s. | q.s. | q.s. | q.s. | q.s. |

Process 1:

Weighed all ingredients.

Solution Phase
1. Taken water for injection (WFI) in a container initially.
2. Added required quantity of Benzalkonium chloride solution in step 1.
3. Added required quantity of Poloxamer in step 2.
4. Added required quantity of Disodium edetate dihydrate in step 3.
5. Added required quantity of Boric acid in step 4.
6. Added required quantity of Sodium chloride in step 5.
7. Added required quantity of Glycerin in step 6.
8. Added required quantity of Propylene glycol in step 7.
9. Optionally, added required quantity of Polyethylene glycol in step 8.
10. To the step 9 WFI added to make batch size to 55% of the final batch size. This is the solution phase.
11. The solution phase is subjected to sterile filtration.

Gel Phase
12. Taken water for injection (WFI) in a container initially.
13. Separately added required quantity of polycarbophil, Povidone and/or Polyvinyl Alcohol to a vessel with continuous stirring and stirred properly till uniform dispersion is observed.
14. Autoclaved step 13 for 121° C. for 20 min.
15. Cool the dispersion at room temperature.

Dispersion Phase
16. Added Loteprednol etabonate (API) to gel phase of step 15 under continuous stirring. Homogenized/milled the suspension using homogenizer/bead mill for appropriate time till desired PSD is achieved.

Final Gel (Dispersion Phase and Solution Phase)
17. Added the dispersion of step 16 to solution phase of step 11 under stirring to get a uniform dispersion.
18. Now pH adjusted to 6.0-7.0.
19. Now made up the weight as per the batch size with WFI under continuous stirring.
20. Filled in approved containers as per marketing requirement.

Process 2:

Weighed all ingredients.

Solution Phase
1. Taken water for injection (WFI) in a container initially.
2. Added required quantity of Benzalkonium chloride solution in step 1.
3. Added required quantity of Disodium edetate dihydrate in step 2.
4. Added required quantity of Boric acid in step 3.
5. Added required quantity of Sodium chloride in step 4.
6. Added required quantity of Glycerin in step 5.
7. Added required quantity of Propylene glycol in step 6.
8. Optionally, added required quantity of Polyethylene glycol in step 7.
9. To the step 8 WFI added to make batch size to 55% of the final batch size. This is the solution phase.
10. The solution phase is subjected to sterile filtration.

Gel Phase
11. Taken water for injection (WFI) in a container initially.
12. Separately added required quantity of polycarbophil, Povidone and/or Polyvinyl Alcohol to a vessel with continuous stirring and stirred properly till uniform dispersion is observed.
13. Autoclaved step 12 for 121° C. for 20 min.
14. Cool the dispersion at room temperature.

Surfactant Phase:
15. Taken water for injection (WFI) in a container initially.
16. Separately added required quantity of Poloxamer in step 15.
17. The surfactant phase is subjected to sterile filtration.

Dispersion Phase
18. Added Loteprednol etabonate (API) to surfactant phase of step 17 under continuous stirring. Homogenized/milled the suspension using homogenizer/bead mill for appropriate time till desired PSD is achieved.

Final Gel (Dispersion Phase, Gel Phase, and Solution Phase)
19. Added the dispersion of step 18 and gel phase of step 14 to solution phase of step 10 under stirring to get a uniform dispersion.
20. Now pH adjusted to 6.0-7.0.
21. Now made up the weight as per the batch size with WFI under continuous stirring.
22. Filled in approved containers as per marketing requirement.

Process 3:

Weighed all ingredients.

Solution Phase
1. Taken water for injection (WFI) in a container initially.
2. Added required quantity of Benzalkonium chloride solution in step 1.
3. Added required quantity of Disodium edetate dihydrate in step 2.
4. Added required quantity of Boric acid in step 3.
5. Added required quantity of Sodium chloride in step 4.
6. Added required quantity of Glycerin in step 5.
7. Added required quantity of Propylene glycol in step 6.
8. Optionally, added required quantity of Polyethylene glycol in step 7.
9. To the step 8 WFI added to make batch size to 55% of the final batch size. This is the solution phase.
10. The solution phase is subjected to sterile filtration.

Gel Phase
11. Taken water for injection (WFI) in a container initially.
12. Separately added required quantity of polycarbophil, Povidone and/or Polyvinyl Alcohol to a vessel with continuous stirring and stirred properly till uniform dispersion is observed.
13. Separately added required quantity of Poloxamer in step 12.
14. Autoclaved step 2 for 121° C. for 20 min.
15. Cool the dispersion at room temperature.

Dispersion Phase
16. Added Loteprednol etabonate (API) to gel phase of step 15 under continuous stirring. Homogenized/milled the suspension using homogenizer/bead mill for appropriate time till desired PSD is achieved.

Final Gel (Dispersion Phase and Solution Phase)
17. Added the dispersion of step 16 to solution phase of step 10 under stirring to get a uniform dispersion.
18. Now pH adjusted to 6.0-7.0.
19. Now made up the weight as per the batch size with WFI under continuous stirring.
20. Filled in approved containers as per marketing requirement.

Stability Studies:

The particle size of the compositions of the invention were tested initially, after 15 days and one month. The compositions of the invention were then observed and tested for particle aggregation, settling, and flocculation on long term storage. The observations at one month, three months, 6 months, 9 months are as follows:

Particle size

| Composition | Time | D₁₀ | D₅₀ | D₉₀ |
|---|---|---|---|---|
| API | Initial | 1.0 | 3.3 | 9.1 |
| Example 5 | Initial | 0.259 | 0.468 | 0.901 |
| | 15 days | 0.253 | 0.470 | 0.900 |
| | 1 month | 0.250 | 0.460 | 0.885 |
| Example 6 | Initial | 0.281 | 0.515 | 1.360 |
| | 15 days | 0.268 | 0.470 | 0.846 |
| | 1 month | 0.272 | 0.476 | 0.883 |
| Example 7 | Initial | 0.253 | 0.489 | 1.280 |
| | 15 days | 0.261 | 0.492 | 0.950 |
| | 1 month | 0.258 | 0.485 | 0.902 |

| | Signs of particle aggregation, settling, and flocculation | | | |
|---|---|---|---|---|
| Composition | One month | Three months | Six months | Nine months |
| Example 1 | No | No | No | No |
| Example 2 | No | No | No | Yes |
| Example 3 | No | No | Yes | Yes |
| Example 5 | No | No | No | No |
| Example 6 | No | No | No | No |
| Example 7 | No | No | No | No |

The compositions of the invention are storage-stable, non-settling, and provide uniform drug delivery from the container. Loteprednol remains effectively suspended in the compositions of the invention for an extended period of time so that the loteprednol is uniformly distributed in the composition. In contrast, non-storage-stable suspensions show signs of particle aggregation, flocculation, and settling or caking at the bottom of container resulting in user instilling an inconsistent and improper dosage.

1. A stable ophthalmic composition comprising therapeutically effective amount of Loteprednol, one or more suspending agents, and one or more pharmaceutically acceptable excipients; wherein the composition is free of non-ionic cellulosic derivatives.

2. The stable ophthalmic composition as claimed in claim 1, wherein the one or more pharmaceutically acceptable excipients are selected from the group comprising viscosity modifier(s), buffering agent(s), chelating agent(s), surfactant(s), tonicity agent(s), preservative(s), or combinations thereof.

3. The stable pharmaceutical composition according to claim 1, wherein the suspending agent is a carboxyvinyl polymer.

4. The stable pharmaceutical composition according to claim 2, wherein the viscosity modifier is selected from the group comprising glycerol, propylene glycol, polyethylene glycol, polyvinyl alcohol, polyvinylpyrrolidone, carbomer, gellan gum, xanthan gum, guar gum, chitosan, alginic acid and its salts, xyloglucan, pectin, hyaluronic acid-agar, carrageenan, shellac, and hyaluronic acid derivatives, dextran, or combinations thereof.

5. The stable pharmaceutical composition according to claim 2, wherein the surfactant is a non-ionic surfactant selected from poloxamers, polysorbates, tyloxapol, or combinations thereof.

6. The stable ophthalmic composition as claimed in claim 1, wherein the composition comprises loteprednol having particle size D₅₀<1 μm and D₉₀<3 μm in an amount of 0.05%-0.5% by weight of the composition.

7. The stable ophthalmic composition as claimed in claim 1, wherein the composition is having a pH in the range of 6.0-7.0.

8. The stable ophthalmic composition as claimed in claim 1, wherein the composition comprises:
Loteprednol in amount of about 0.05% w/w to about 0.5% w/w of the pharmaceutical composition;
suspending agent(s) in amount of about 0.1% w/w to about 5.0% w/w of the pharmaceutical composition;
viscosity modifier(s) in amount of about 0.1% w/w to about 5% w/w of the pharmaceutical composition;
preservative(s) in an amount of about 0.001% w/w to 0.05% w/w of the pharmaceutical composition;
surfactant(s) in an amount of about 0.05% w/w to 1.0% w/w of the pharmaceutical composition;
chelating agent(s) in amount of about 0.01% w/w to about 0.1% w/w of the pharmaceutical composition;
tonicity adjusting agent(s) in an amount of about 0.01% w/w to about 0.5% w/w of the pharmaceutical composition,
wherein the composition is having a pH in the range of 6.0-7.0.

9. The stable ophthalmic composition as claimed in claim 8, wherein the composition is a suspension which is storage stable with no signs of particle aggregation or flocculation for at least six months.

10. The stable ophthalmic composition as claimed in claim 9, wherein the composition comprises:
Loteprednol etabonate in amount of about 0.38% w/w of the pharmaceutical composition;
carboxyvinyl polymer in amount of about 0.375% w/w of the pharmaceutical composition;
viscosity modifiers selected from glycerol, propylene glycol, polyethylene glycol, polyvinyl alcohol, polyvinylpyrrolidone or combinations thereof, in amount of about 0.5% w/w to about 3% w/w of the pharmaceutical composition;
benzalkonium chloride in an amount of about 0.003% w/w of the pharmaceutical composition;
poloxamer 407 in an amount of about 0.2% w/w of the pharmaceutical composition;
edetate disodium in amount of about 0.055% w/w of the pharmaceutical composition;
sodium chloride in an amount of about 0.05% w/w of the pharmaceutical composition,
wherein the composition is having an initial pH of 6.5±0.2.
